(19) United States
(12) Patent Application Publication
(10) Pub. No.: US 2012/0078329 A1
(43) Pub. Date: Mar. 29, 2012

(54) OPTICAL TREATMENT APPARATUS

(75) Inventor: Takuo Shimada, Kanagawa (JP)

(73) Assignee: PANASONIC CORPORATION, Osaka (JP)

(21) Appl. No.: 13/241,785

(22) Filed: Sep. 23, 2011

(30) Foreign Application Priority Data
Sep. 27, 2010 (JP) 2010-214885

(51) Int. Cl. A61N 5/06

(52) U.S. Cl. 607/88

(57) ABSTRACT

It is an object of the invention to provide an optical treatment apparatus having a light emitting surface which can properly carry out an optical treatment of the treating apparatus continuously while reducing a temperature of the light emitting surface. There is provided an optical treatment apparatus including surface irradiating means having a plurality of light emitting modules connected in series, and current supplying means for supplying a current to the surface irradiating means. The light emitting module has a light emitting device and an individual current control device which are connected to each other in parallel. The individual current control device is configured to have a greater resistance value than a load impedance of the light emitting device at a predetermined temperature or less and to have a smaller resistance value than the load impedance at a temperature exceeding the predetermined temperature.

### CROSS REFERENCE TO RELATED APPLICATIONS

This application is entitled to the benefit of Japanese Patent Application No. 2010-214855, filed on Sep. 27, 2010, the disclosure of which including the specification, drawings and abstract is incorporated herein by reference in its entirety.

### TECHNICAL FIELD

The present invention relates to an optical treatment apparatus for irradiating a light onto an affected portion, thereby relieving a pain of a muscle and joint tissue or suppressing an inflammation.

### BACKGROUND ART

Conventionally, there is devised and practically applied an optical treatment apparatus for irradiating an affected portion (for example, a hand, a wrist, a foot, an ankle, a knee and the like) with a light emitted from a light emitting device disposed in contact with the affected portion or in the vicinity thereof, thereby relieving a pain of a muscle and joint tissue or suppressing an inflammation (for example, see Patent Literature 1 and Patent Literature 2). In the optical treatment apparatus, a near infrared high output LED having excellent organism permeability is used as a light emitting device. By laying flatly a large number of LEDs connected each other in series and/or parallel, it is possible to increase an area of a light emitting region, to enhance a treating effect, and to shorten a time required for a treatment.

In the case where the high output near infrared light emitting device is arranged to be close to or to come in contact with an affected portion, thereby carrying out an optical treatment, however, there is also a possibility that a redness or a low temperature burn injury of the affected portion might be generated with a rise in a skin temperature. Therefore, there is also proposed a mechanism in which temperature detecting means for monitoring a temperature in an LED or in the vicinity of a light emitting surface thereof is provided and in which a light irradiation is stopped if a detected temperature exceeds a predetermined temperature (for example, see Patent Literature 3 and Patent Literature 4).

### CITATION LIST

### Patent Literature

[Patent Literature 1] Japanese Patent Application Laid-Open No. 11-192315
[Patent Literature 2] Japanese Patent Application Laid-Open No. 2001-187159
[Patent Literature 3] Japanese Patent Application Laid-Open No. 02-246988
[Patent Literature 4] Japanese Patent Application Laid-Open No. 2008-022894

### SUMMARY OF INVENTION

### Technical Problem

Referring to an optical treatment apparatus having a structure according to the related art, however, when the temperature detecting means detects that a temperature of an LED in a specific place or in the vicinity of a light emitting surface thereof exceeds a predetermined temperature during a light irradiation, the light irradiation over the whole treating apparatus is stopped. For this reason, there is a problem that an optical treatment cannot be carried out continuously.

The rise in the temperature which exceeds the predetermined temperature occurs due to a partial continuous contact of a light emitting surface with an irradiated body or due to a defective heat radiation more often as compared with a failure of the LED itself. In other words, a temperature in only a part of the light emitting surface is raised to be equal to or higher than the predetermined temperature. More specifically, the temperature in only a part of the light emitting surface tends to be raised to be equal to or higher than the predetermined temperature in the case that 1) a light irradiation is continuously carried out for a long period of time while closely contacting a skin surface convex portion to be an irradiated body with a part of the light emitting surface, 2) a large number of hairs, moles, tattoos or pigmentation parts are present in a part of a skin to be an irradiated body so that an absorption of a heat is facilitated, 3) an irradiation is continuously carried out over a long period of time while attaching a foreign substance intercepting a light to a light emitting surface, or 4) a ring, a watch or the like is worn by patient.

It is not preferable a stoppage of a lighting operation of a whole light source in the optical treatment apparatus in the case that the temperature in only a part of the light emitting surface is raised, because of interrupting an optical treatment. Therefore, it is an object of the present invention to provide an optical treatment apparatus capable of properly carrying out an optical treatment itself of the treating apparatus continuously, while reducing a temperature in only a part of a light emitting surface when the temperature in that part is raised.

### Solution to Problem

In order to achieve the object, the optical treatment apparatus according to the present invention includes surface irradiating means in which a plurality of light emitting modules is connected in series each other and current supplying means for supplying a current to the surface irradiating means. The light emitting module has a light emitting device and an individual current control device which are connected to each other in parallel. The individual current control device is configured to have a greater resistance value than a load impedance of the light emitting device at a predetermined temperature or less, and to have a smaller resistance value than the load impedance of the light emitting device at a temperature exceeding the predetermined temperature. Consequently, the desired object can be achieved.

### Advantageous Effects of Invention

The optical treatment apparatus according to the present invention can properly carry out a treatment thereof continuously, while interrupting a light irradiation from a part of the light emitting modules included in the surface irradiating means when the light emitting module has a temperature which is equal to or higher than a predetermined temperature. In other words, in the optical treatment apparatus according to the present invention, the individual current device can control ON/OFF of a light emission for each light emitting module. Consequently, it is possible to continuously carry out a proper optical treatment.

### BRIEF DESCRIPTION OF DRAWINGS

FIG. 1 is a circuit block diagram showing an optical treatment apparatus according to Embodiment 1 of the present invention;

FIG. 2 is a chart showing characteristics of a temperature and a resistance value of a CTR (Critical Temperature Coefficient) thermistor of the optical treatment apparatus according to Embodiment 1 of the present invention;

FIG. 3 is a perspective view showing a structure of a light emitting module in the optical treatment apparatus according to Embodiment 1 of the present invention;

FIG. 4 is a circuit block diagram showing an optical treatment apparatus according to Embodiment 2 of the present invention;

FIG. 5 is a perspective view showing planar irradiating means in which a large number of light emitting modules are disposed to configure a surface light source according to Embodiment 2 of the present invention; and

FIG. 6 is a circuit block diagram showing an optical treatment apparatus according to Embodiment 3 of the present invention.

### DESCRIPTION OF EMBODIMENTS

The optical treatment apparatus according to the present invention includes 1) surface irradiating means having a plurality of light emitting modules and 2) current supplying means.

The surface irradiating means is a member having a light emitting surface and serves to irradiate an affected portion with a light from the light emitting surface, thereby carrying out an optical treatment. A light sent from the light emitting surface is emitted from the light emitting modules possessed by the surface irradiating means. The current supplying means supplies a current to the surface irradiating means so as to feed a necessary current for the light emission from each of the light emitting modules.

The light emitting modules possessed by the surface irradiating means are electrically connected to each other in series. All of the light emitting modules possessed by the surface irradiating means may be connected in series in a line (Embodiments 1 and 3 which will be described below) or the light emitting modules possessed by the surface irradiating means may be divided into some light emitting module groups and each of the light emitting module groups may be connected in series in a line (Embodiment 2 which will be described below). The light emitting module groups connected in series in a line will be referred to as a “light emitting module assembly”. At this time, the light emitting module assemblies are electrically connected to each other in parallel (Embodiment 2).

The light emitting module assemblies possessed by the surface irradiating means may have a current limiting device connected to the light emitting modules contained therein in series (Embodiment 2). The current limiting device is an overcurrent protecting device for preventing an overcurrent exceeding a rated current from flowing. The current limiting device is not particularly restricted, and a device using a power IC, a device using a nonlinear resistance device such as a so-called resettable fuse or the like can be used as the current limiting device.

Each of the light emitting modules possessed by the surface irradiating means has a light emitting device and an individual current control device, and the light emitting device and the individual current control device are electrically connected in parallel. The light emitting device is not particularly restricted, and an LED light emitting device is preferable and a near infrared light emitting diode is more preferable. More specifically, the individual current control device is a resistor having an electrical resistance varied with a change in a temperature and is also referred to as a thermistor.

Each of the light emitting modules has a light emitting device and an individual current control device which are connected to each other in parallel as described above. It may have a single light emitting device and a single individual current control device (Embodiments 1 and 2 which will be described below), and may have a plurality of light emitting devices and a single individual current control device (Embodiment 3 which will be described below).

As described above, the individual current control device is typically a thermistor. The individual current control device has a greater resistance value than a load impedance of the light emitting device connected to the individual current control device in parallel at a predetermined temperature or less. On the other hand, the individual current control device has a smaller resistance value than the load impedance of the light emitting device connected to the individual current control device in parallel at a temperature exceeding the predetermined temperature.

The thermistor is generally divided roughly into a thermistor in which a resistance value is decreased with a rise in a temperature (a CTR thermistor or an NTC thermistor) and a thermistor in which a resistance value is increased with a rise in a temperature (a PTC thermistor). The individual current control means of the optical treatment apparatus according to the present invention is a thermistor in which a resistance value is decreased with a rise in a temperature.

Furthermore, it is preferable that the thermistor to be the individual current control device should be a CTR (Critical Temperature Coefficient) thermistor. The CTR thermistor is generally made of a vanadium oxide based material and is also referred to as a sudden change thermistor. The CTR thermistor has a Curie temperature to be a predetermined phase transition point where a crystal structural is changed, and has a characteristic that a resistance is suddenly decreased when a temperature thereof exceeds Curie temperature. For example, the resistance is suddenly decreased in order of 100Ω to 0.1Ω. For this reason, the CTR thermistor is generally utilized as a heat switch for detecting a heat generation in a short time.

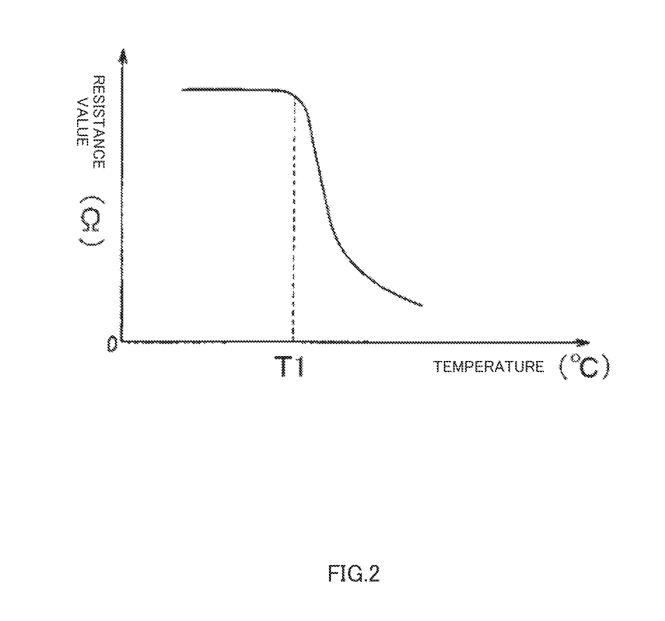

FIG. 2 is a graph showing a relationship between a resistance value (a Y axis) and a temperature (an X axis) in the CTR thermistor 9. It is apparent that the resistance value is suddenly decreased when a temperature exceeds T1. Temperature T1 is referred to as the Curie temperature.

Moreover, Curie temperature T1 of the CTR thermistor can be adjusted by adding an oxide of metal such as tungsten, germanium, iron and so on, to VO₂—BaO—P₂O₅ based composite oxide sintered body constituting the CTR.

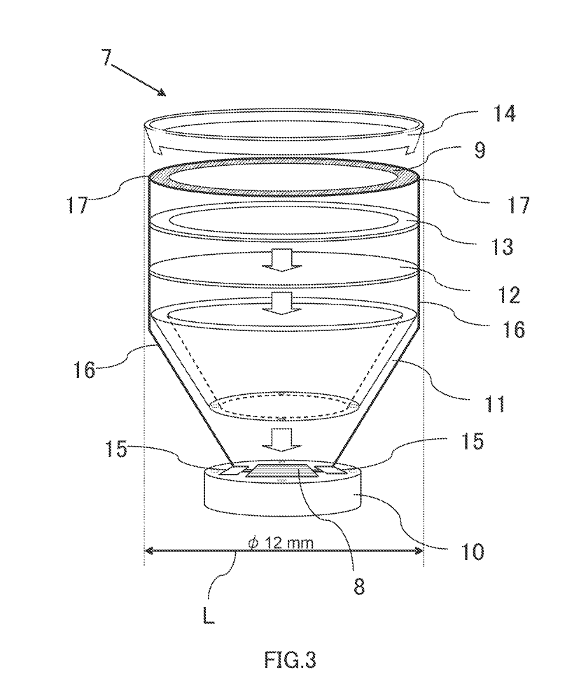

In the optical treatment apparatus according to the present invention, it is preferable that the individual current control device should be provided in the vicinity of an irradiating target surface of a light emitting device which is connected in parallel with the individual current control device (see FIG. 3). The irradiating target surface of the light emitting device is a surface (a light emitting surface) with which an affected portion to be optically treated can come in contact. By disposing the individual current control device in the vicinity of the irradiating target surface of the light emitting device, it is possible to make a temperature of the individual current control device to approach to that of the affected portion.

Furthermore, it is preferable that the individual current control device should be disposed in a position where a light sent from the light emitting device is not directly irradiated (see FIG. 3). When the light sent from the light emitting device is irradiated, a temperature is raised. Consequently, a difference between the temperature of the individual current control device and that of the affected portion increases.

More specifically, the individual current control device can be disposed in an outer peripheral part of a region which is irradiated with the light from the light emitting device. The region which is irradiated with the light from the light emitting device may be defined by a light reflecting mirror (see FIG. 3). Therefore, it is preferable to dispose the individual current control device in the outer peripheral part of the light irradiating region thus defined.

Moreover, a heat insulator may be provided between the individual current control device and the light emitting device (see FIG. 3). It is possible to more effectively prevent the individual current control device from being heated with the light sent from the light emitting device by providing the heat insulator therebetween. Thus, is easy to make the temperature of the individual current control device to approximate to that of the affected portion.

By the optical treatment apparatus according to the present invention, the optical treatment is carried out by contacting the affected portion with the light emitting surface of the surface irradiating means (or the irradiating target surface of the light emitting device). There is a possibility that a redness or a low temperature burn injury might be generated in the affected portion when a temperature of the contact part of the affected portion with the light emitting surface exceeds a “predetermined temperature” during the treatment.

The “predetermined temperature” is not particularly restricted, and it is generally apparent that there is a risk of the generation of the low temperature burn injury in a case that a skin surface of a human body is heated continuously for approximately 360 minutes at 44° C. It is apparent the time duration for the risk of the onset of low temperature burn injury halves as the heating temperature rises by 1° C. (that is, approximately 180 minutes at 45° C., approximately 90 minutes at 46° C., and approximately six minutes at 50° C.). Accordingly, the “predetermined temperature” in the optical treatment apparatus according to the present invention should be set properly depending on an executing situation such as a treating time required for an optical treatment or a light output power, and is preferably equal to or lower than 50° C. in respect of a safety.

For the cause of the temperature of the contact part of the affected portion with the light emitting surface to exceed the “predetermined temperature,” the following can be supposed. More specifically, a large number of hairs, moles, tattoos, pigmentation parts or the like are present in a part of the affected portion (for example, the skin surface) to be an irradiated body, and the affected portion excessively absorbs a heat of a light sent from the light emitting surface. As a result, the temperature is raised. Thus, in the case where the temperature of the contact part of the affected portion with the light emitting surface exceeds the “predetermined temperature,” only a part of the contact part selectively exceeds the “predetermined temperature” and the other portions are maintained to have the predetermined temperature or less.

The optical treatment apparatus according to the present invention can selectively prevent a light from being sent to the contact part of the affected portion with the light emitting surface, thereby reducing a temperature when the temperature of the contact part exceeds the “predetermined temperature” during the optical treatment. In other words, it is possible to suppress a light emission from only a part of the light emitting modules possessed by the surface irradiating means.

As described above, the surface irradiating means in the optical treatment apparatus according to the present invention includes the light emitting module having the light emitting device and the individual current control device which are connected to each other in parallel. A resistance value of the individual current control device at the predetermined temperature is adjusted. Consequently, a current flows to the light emitting device so as to generate a light emission when the affected portion has the predetermined temperature or less, and the current can not flow to the individual current control device so as not to generate the light emission when the affected portion has the predetermined temperature or less.

Furthermore, the surface irradiating means in the optical treatment apparatus according to the present invention has a plurality of light emitting modules connected to each other in series. In the case that the current is not supplied to the light emitting device and supplied to the individual current control device in some of the light emitting modules, the light emission is not generated in the some of the light emitting module. Even in the case, the current is supplied to the other light emitting modules. Therefore, it is possible to continuously carry out the light emission in the other light emitting modules.

In the optical treatment apparatus according to the present invention, thus, a light irradiation on “a portion exceeding the predetermined temperature” in the contact part of the affected portion with the light emitting surface is selectively suppressed, and the light irradiation on “a portion maintained to have the predetermined temperature or less” is continuously carried out. If it is possible to continuously irradiate “the portion maintained to have the predetermined temperature or less” in the contact part, the optical treatment itself can be consecutively carried out by the optical treatment apparatus. Therefore, the optical treatment apparatus according to the present invention has a convenience.

An embodiment of an optical treatment apparatus according to the present invention will be described below in detail with reference to the drawings.

### Embodiment 1

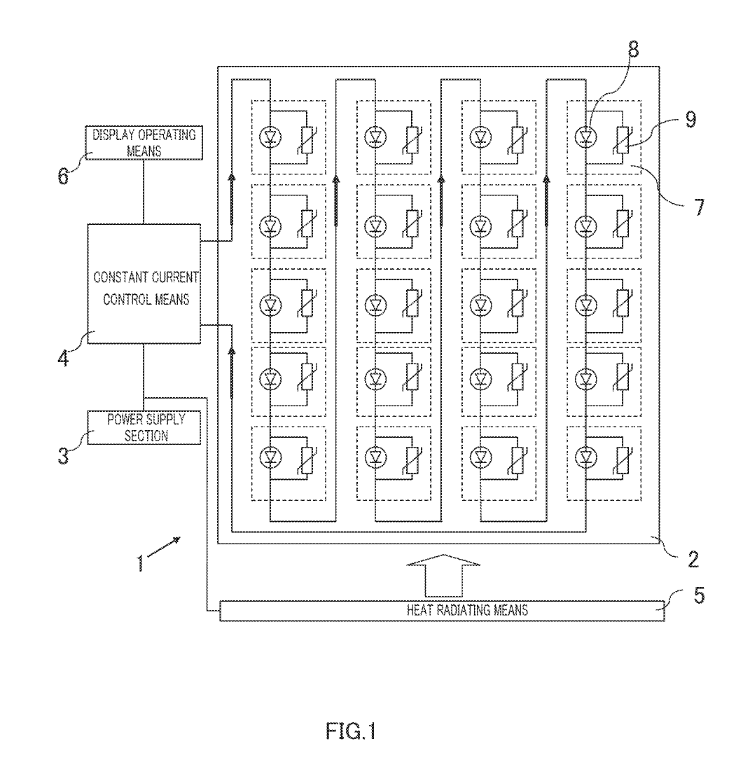

FIG. 1 is a circuit block diagram showing an optical treatment apparatus 1 according to Embodiment 1 of the present invention. Optical treatment apparatus 1 shown in FIG. 1 includes surface irradiating means 2, power supply section 3, constant current control means 4, heat radiating means 5 and display operating means 6.

Surface irradiating means 2 is used as a surface light source. Surface irradiating means 2 includes a single large area reflecting board (for example, an aluminum board) and 20 light emitting modules 7 attached onto the board. 20 light emitting modules 7 are connected to each other in series.

Each light emitting module 7 has near infrared high output LED 8 to be a light emitting device and CTR thermistor 9 to be an individual current control device, and both of them are integrated with each other. LED 8 and CTR thermistor 9 are electrically connected to each other in parallel.

Each LED 8 emits a light of wavelength of 830 nm and of luminous flux of 5 W. Surface irradiating means 2 has a light emitting area of approximately 200 cm². LEDs 8 are arranged so that an average light power density of a light emitting surface of surface irradiating means 2 is 0.5 W/cm².

As described above, CTR thermistor 9 has a characteristic that a resistance is suddenly decreased when a temperature thereof exceeds Curie temperature T1 (see FIG. 2).

Power supply section 3 supplies a constant current to all of light emitting modules 7 of surface irradiating means 2 through constant current control means 4. Constant current control means 4 and power supply section 3 may have such a structure as to form current supplying means with an integral construction.

Heat radiating means 5, to which a voltage from power supply section 3 is supplied, serves to cool surface irradiating means 2. Heat radiating means 5 specifically includes a cooling fan and a radiation fin, and is always driven under applying current. It is suitable to have mechanism in which heat radiating means 5 independently includes an internal cooling system and an external cooling system; the internal cooling system being for suppressing a heat generation with an ON operation of LED 8, and the external cooling system for exposing cooling air to the whole light emitting surface from an external side surface in such a manner that an irradiating port does not undergo self-heating by an influence of a passing current of the CTR thermistor 9 (which is not shown).

Display operating means 6 carries out a light irradiation control of ON/OFF of a light irradiation on surface irradiating means 2 through constant current control means 4 in accordance with an instruction of an operator.

In the optical treatment apparatus configured as shown in FIG. 1, a constant current is continuously supplied to light emitting module 7 through constant current control means 4 so that an affected portion can be irradiated with a light through the light emitting surface from surface irradiating means 2.

In some cases that only a part of the light emitting modules 7 excessively undergoes a heat so that temperature thereof exceeds a predetermined temperature while the affected portion is irradiated with the light from surface irradiating means 2. The light emitting module 7 which excessively undergoes a heat beyond the predetermined temperature will be referred to as “specific light emitting module 7′.” As described above, a cause for specific light emitting module 7′ to exceed the predetermined temperature can be supposed that an excessive heat absorption progresses over a part of an irradiated subject (for example, a skin of a patient).

When a temperature of specific light emitting module 7′ exceeds the predetermined temperature, a current flows to CTR thermistor 9 of specific light emitting module 7′ without the flow of the current to LED 8, so that lighting can be stopped. Thus, a state in which the current flows to CTR thermistor 9 and does not flow to LED 8 is referred to as a “dummy short-circuit state.” Even in the case that specific light emitting module 7′ is brought into the dummy short-circuit state, a constant current is supplied to the other light emitting modules 7 which are connected in series with specific light emitting module 7′. Therefore, the light emission of the LED 8 of the other light emitting modules 7 can be carried out continuously.

Description will be given to the reason why the lighting of LED 8 of specific light emitting module 7′ is stopped and the reason why the light emission of the other light emitting modules 7 is carried out continuously.

As described above, light emitting module 7 has LED 8 serving as the light emitting device and CTR thermistor 9 serving as the individual current control device, and both of them are electrically connected in parallel and are integrated with each other. In the case that the temperature of CTR thermistor 9 is equal to or lower than Curie temperature T1 (see FIG. 2), a load impedance (a forward voltage/a current ratio) of LED 8 is considerably lower than a resistance value of CTR thermistor 9. Therefore, most of the current flows to LED 8 so as to emit light.

On the other hand, when a temperature of specific light emitting module 7′ is raised to exceed Curie temperature T1, the resistance value of CTR thermistor 9 of specific light emitting module 7′ is reduced suddenly. As a result, a bypass of the current is formed so that the current does not flow to LED 8 of specific light emitting module 7′ so that LED 8 is turned OFF. This state represents the dummy short-circuit state.

Even when specific light emitting module 7′ is brought into the dummy short-circuit state, the current is supplied to light emitting modules 7 other than specific light emitting module 7′. For this reason, LED 8 of light emitting module 7 other than specific light emitting module T continuously emits a light. Therefore, it is possible to continuously irradiate with a light without stopping the optical treatment.

Thus, by setting Curie temperature (T1) of CTR thermistor 9 to be a temperature (a predetermined temperature) at which the light emission of LED 8 should be stopped so as to turn OFF, it is possible to reliably control the ON/OFF operation of LED 8.

Although the predetermined temperature (the Curie temperature of the CTR thermistor) is set depending on an executing situation such as a treatment time required for the optical treatment apparatus or an output of a light as described above, it is preferable that the predetermined temperature should be equal to or lower than 50° C. in respect of a safety.

In CTR thermistor 9, the resistance value is suddenly decreased when a temperature thereof reaches the Curie temperature. When a rise in the temperature up to the Curie temperature or more is observed, therefore, it is possible to turn OFF LED 8 with a high sensitivity. For this reason, the thermistor is preferable for the individual current control device of the optical treatment apparatus in respect of a safety of a patient.

Moreover, the resistance value of CTR thermistor 9 is suddenly decreased when the temperature thereof become Curie temperature T1 or more. For this reason, a heat is hard to generate while the current flows to CTR thermistor 9. Consequently, it is possible to reduce a current consumption by using the CTR thermistor for the individual current control device of the optical treatment apparatus.

Next, a structure of light emitting module 7 will be described with reference to FIG. 3. Light emitting module 7 shown in FIG. 3 includes individual heat radiating board 10 made of aluminum and LED 8 mounted on a surface of individual heat radiating board 10.

It is preferable that individual heat radiating board 10 should be attached in close contact with a large area reflecting board (for example, an aluminum board) of surface irradiating means 2 (see FIG. 1). The reason is that a heat generated from light emitting module 7 can be conducted to the large area aluminum board to promote a heat radiation.

Reflecting mirror 11 is provided on individual heat radiating board 10. The reflecting mirror 11 is composed of an inverted conical cylinder-shaped container having an internal wall surface deposited with aluminum. The inverted conical cylinder-shaped container can be made of resin. A light emitted from LED 8 is emitted upward, and a light irradiating region thereof is defined by reflecting mirror 11. Diameter L of an opening portion on an upper surface in reflecting mirror 11 is not particularly restricted, and it can be set to be approximately 12 mm.

Transparent disk 12 is disposed on the opening portion of the upper surface in reflecting mirror 11. Transparent disk 12 is a light irradiating target surface of LED 8, and prevents a dust from attaching to LED 8 or a ingress of water to LED 8 while transmitting a light irradiated from LED 8 without a loss. A shape of transparent disk 12 is not restricted to a flat plate, and may be an aspherical lens for collimating a beam. And also, a light diffusing material for causing a light intensity distribution to be uniform can also be used as material for transparent disk 12.

Ring-shaped CTR thermistor 9 is provided on an inward part of an outer peripheral edge of transparent disk 12 via heat insulating cushion 13 composed of a ring-shaped sponge. Heat insulating cushion 13 thermally separates LED 8 from CTR thermistor 9. A white color based sheet can be lined on a surface (a lower surface in FIG. 3) of heat insulating cushion 13 on which CTR thermistor 9, and thereby CTR thermistor 9 is difficult to be influenced by a radiation heat leaked from an outer periphery of reflecting mirror 11.

Furthermore, presser ring 14 is provided on CTR thermistor 9. Presser ring 14 is configured by a material having a high thermal conductivity, for example, a metal. Presser ring 14 is thermally coupled to CTR thermistor 9. Presser ring 14 serves to maintain a mechanical strength, and furthermore, to transmit a temperature of a subject such as a skin of a human body to CTR thermistor 9.

Thus, in light emitting module 7 of FIG. 3, heat insulating cushion 13, CTR thermistor 9 and presser ring 14 are disposed on an outside of the internal wall surface of reflecting mirror 11. That is, they are arranged at an outer peripheral portion of a light irradiating region of a light emitted from LED 8. For this reason, heat insulating cushion 13, CTR thermistor 9 and presser ring 14 are not irradiated the light emitted from LED 8, and are not directly heated by the light.

On the other hand, CTR thermistor 9 is disposed in the vicinity of the irradiating target for LED 8, and furthermore, is provided in contact with presser ring 14. Therefore, a temperature of CTR thermistor 9 easily approximates to that of presser ring 14. Moreover, presser ring 14 contacts with the affected portion. Therefore, the temperature of presser ring 14 tends to approximate to that of the affected portion. Accordingly, the resistance value of CTR thermistor 9 tends to be depended on the temperature of the affected portion. For this reason, ON/OFF of light emitting module 7 is controlled based on a temperature of the affected portion which is to be irradiated with the light.

Electrodes 15 are led from an anode and a cathode in LED 8, respectively. Electrode 15 and CTR thermistor 9 are electrically connected to each other in parallel through lead wire 16. Thus, a single electrical equivalent circuit of light emitting module 7 is obtained by simply connecting LED 8 and CTR thermistor 9 to each other in parallel.

In light emitting module 7 in FIG. 3, CTR thermistor 9 and electrical connecting section 17 are molded in such a manner that LED 8 is not broken due to an application of an electrostatic noise to lead wire 16 from an outside. Furthermore, an inductor device may be inserted, or other overcurrent or overvoltage/backward voltage protecting device may be provided, in such a manner that a spontaneous large current exceeding a rated value does not flow to LED 8 through lead wire 16.

An anode electrode (electrode 15) of one light emitting module 7 and a cathode electrode (electrode 15) of an another light emitting module 7 adjacent to the one light emitting module are cascade connected in series. Consequently, a large number of light emitting modules 7 can be laid over the large area reflecting board of surface irradiating means 2 (see FIG. 1).

Specific light emitting module 7′ in the optical treatment apparatus having the structure described above is brought into the dummy short-circuit state when the temperature of CTR thermistor 9 approximating to the temperature of the affected portion to be irradiated exceeds Curie temperature (T1). A current does not flow to LED 8 of specific light emitting module 7′ falling into the dummy short-circuit state so that the light emission is stopped. On the other hand, the light emission of LED 8 in light emitting module 7 other than specific light emitting module 7′ is continuously carried out so that the optical treatment apparatus can consecutively perform the treatment.

In specific light emitting module 7′ falling into the dummy short-circuit state, moreover, in the case where the temperature of CTR thermistor 9 (the surface temperature of the affected portion) is reduced to a proper temperature, the dummy short-circuit state is resolved so that a suppression in the light emission of LED 8 is also cancelled automatically and a return to a normal lighting operation is automatically carried out again.

In the optical treatment apparatus according to the present embodiment, thus, it is possible to obtain both of consistency of a maintenance of a safety and treatment effect for the affected portion which is provided in close contact with or close to an irradiating port. In addition, it is possible to provide an optical treatment apparatus having a safe and an efficient temperature management for each portion of a light emitting surface even if a complicated control circuit or the like is not disposed.

### Embodiment 2

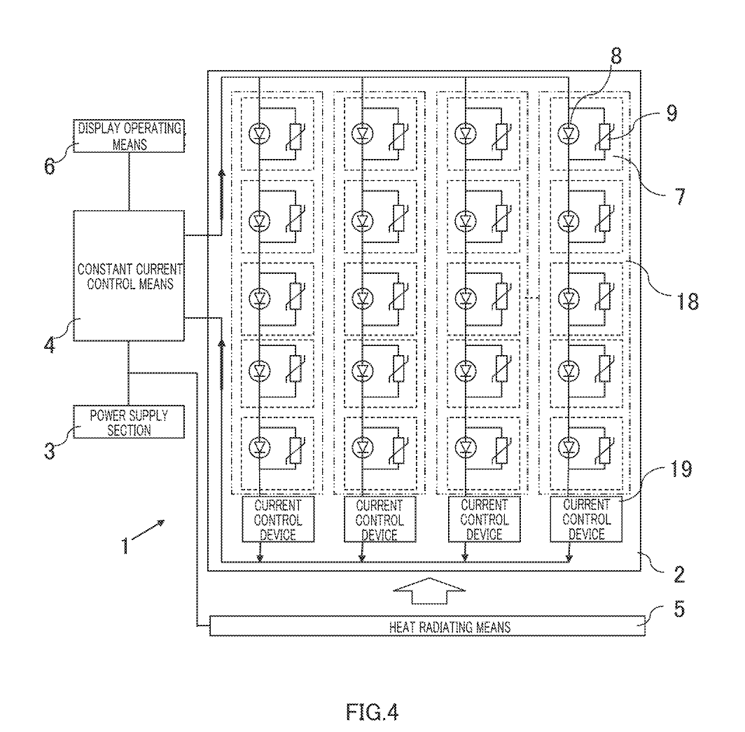

FIG. 4 is a circuit block diagram showing Embodiment 2 of optical treatment apparatus 1 according to the present invention. In Embodiment 2, the same components as those described with reference to FIG. 1 according to Embodiment 1 have the same reference numerals and description will be omitted.

A circuit block according to Embodiment 2 has a feature that a plurality of light emitting module assemblies 18 having a plurality of light emitting modules 7 connected in series is connected in parallel.

In order to increase an area of a light emitting surface of surface irradiating means, a larger number of light emitting modules 7 are required. As shown in the circuit block diagram of FIG. 1 according to Embodiment 1, a high voltage is required to be supplied when all of light emitting modules 7 are connected in series. In Embodiment 2, light emitting module assemblies 18 having light emitting modules 7 connected in series are connected in parallel in order to avoid the supply of the high voltage.

In some cases where a plurality of light emitting module assemblies 18 are connected in parallel each other, however, passing currents of respective light emitting module assemblies 18 are greatly different from each other due to a variation in a forward voltage characteristic for each LED 8. Consequently, there is a possibility that a current exceeding a rated current might be supplied to LED 8 included in specific light emitting module assembly 18. Therefore, it is preferable that current limiting device 19 with a power IC should be connected in series to apply limitation of the current every light emitting module assembly 18. By disposing current limiting device 19, thus, it is possible to easily achieve an increase in the area of light emitting module 7, thereby enhancing an extensibility of the optical treatment apparatus even if a constant current control is not taken for each light emitting module assembly 18.

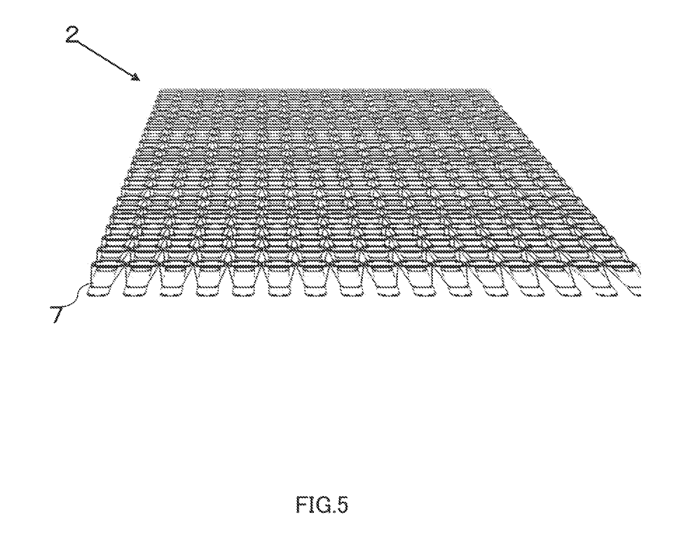

FIG. 5 is a perspective view showing surface irradiating means 2 in which a large number of light emitting modules 7 are disposed to form a surface light source. The number of light emitting modules 7 which are disposed is not limited to 20, and may be at least 100.

### Embodiment 3

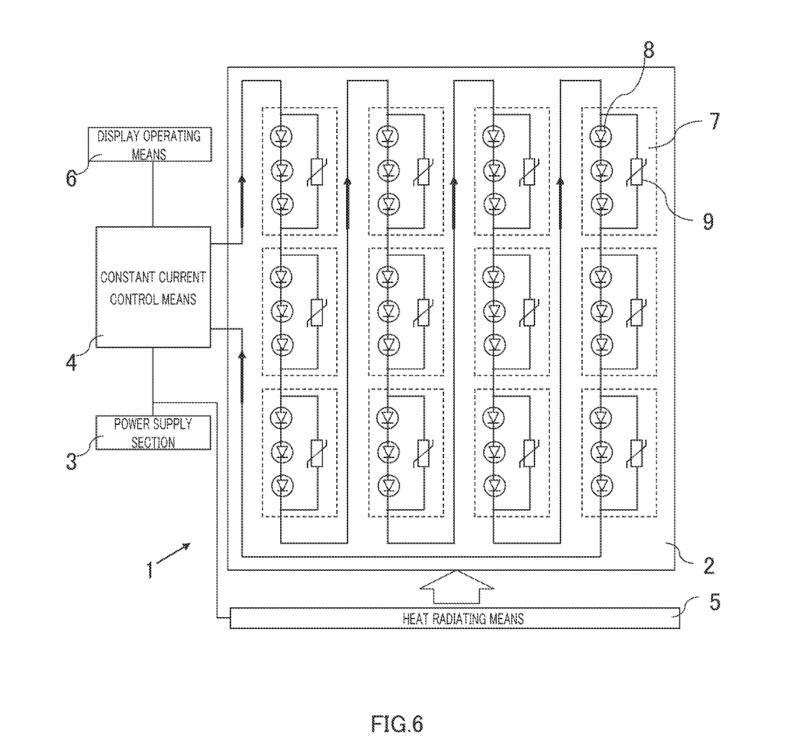

FIG. 6 is a circuit block diagram showing Embodiment 3 of optical treatment apparatus 1 according to the present invention. In Embodiment 3, the same components as those described in Embodiments 1 and 2 have the same reference numerals, and description will be omitted.

With the structures according to Embodiments 1 and 2, each of light emitting modules 7 has a single individual current control device connected to a single LED in parallel. On the other hand, in the structure according to Embodiment 3, light emitting module 7 has a single individual current control device (CTR thermistor 9) connected in parallel with a plurality of LEDs which is connected in series. The structure according to Embodiment 3 is different from that according to Embodiment 1 in this respect and is identical thereto in the other respects.

In the circuit block diagram shown in FIG. 6, 12 light emitting modules 7 are connected in series. Each of light emitting modules 7 has single CTR thermistor 9 connected in parallel with three LEDs 8 which are connected to each other in series.

In optical treatment apparatus 1 shown in the circuit block diagram of FIG. 6, when CTR thermistor 9 of specific light emitting module 7′ among light emitting modules 7 has a predetermined temperature or more, specific light emitting module 7′ is brought into a dummy short-circuit state. As a result, three LEDs 8 of specific light emitting module 7′ which is set into the dummy short-circuit state stops lighting. On the other hand, a constant current is supplied to light emitting modules 7 other than specific light emitting module 7′. For this reason, LEDs 8 of other light emitting modules 7 continuously emit a light.

In specific light emitting module 7′ falling into the dummy short-circuit state, moreover, in the case where the temperature of CTR thermistor 9 (a surface temperature of an affected portion) is reduced to a proper temperature, the dummy short-circuit state is also resolved so that a suppression in the light emission of three LEDs 8 is automatically canceled and a return to a normal lighting operation is automatically carried out again.

With the structure according to Embodiment 3, ON/OFF control for each LED is not taken, which is differently from Embodiments 1 and 2. With the structure according to Embodiment 3, a plurality of LEDs is formed into a single block and the ON/OFF control for each block is taken. According to the structure in accordance with Embodiment 3, it is possible to reduce the number of components of CTR thermistor 9. Therefore, the structure according to Embodiment 3 is particularly effective for an application to an optical treatment apparatus having a large number of light emitting modules 7.

Although all of light emitting modules 7 included in surface irradiating means are connected in series in Embodiment 3 in the same manner as in Embodiment 1, furthermore, it is apparent that a light emitting module assembly having light emitting modules 7 connected in series may be connected in parallel in the same manner as in Embodiment 2.

According to the optical treatment apparatuses having the structures in accordance with Embodiments 1 to 3, in the case where the light emitting surface of surface irradiating means 2 is contacted with the affected portion (for example, a knee joint or the like) for a long period of time and where a temperature of only a part of the light emitting surface is raised to be a predetermined temperature or more, only LED 8 for irradiating the part can be turned OFF to reduce the temperature of the same part and other LEDs 8 can be continuously turned ON. According to the optical treatment apparatuses, therefore, it is possible to execute an efficient treatment while ensuring a safety of a patient.

### INDUSTRIAL APPLICABILITY

The optical treatment apparatus according to the present invention includes surface irradiating means having a plurality of light emitting modules. And the optical treatment apparatus can selectively turns OFF only a light emitting device in any of the light emitting modules which has a higher temperature than a predetermined temperature during an optical treatment, and can continuously turns ON light emitting devices of the other light emitting modules which have temperatures same as or less than the predetermined temperature. Therefore, the optical treatment apparatus according to the present invention can continuously carry out an optical treatment properly. Consequently, the present invention can be expected to be widely utilized as an optical treatment apparatus for relieving a pain of a muscle and joint tissue or suppressing an inflammation.

### REFERENCE SIGNS LIST

1 optical treatment apparatus
2 surface irradiating means
3 power supply section
4 constant current control means
5 heat radiating means
6 display operating means
7 light emitting module
8 LED
9 CTR thermistor
10 individual heat radiating board
11 reflecting mirror
12 transparent disk
13 heat insulating cushion
14 presser ring
15 electrode
16 lead wire
17 electrical connecting section
18 light emitting module assembly
19 current limiting device

1. An optical treatment apparatus comprising: a surface irradiating section that has a plurality of light emitting modules connected in series each other; and a current supplying section that supplies a current to the surface irradiating section, wherein
the light emitting module has a light emitting device and an individual current control device which are connected to each other in parallel, and
the individual current control device has a greater resistance value than a load impedance of the light emitting device at a predetermined temperature or less, and has a smaller resistance value than the load impedance of the light emitting device at a temperature exceeding the predetermined temperature.

2. An optical treatment apparatus comprising: a surface irradiating section that has a plurality of light emitting modules connected in series each other; and a current supplying section that supplies a current to the surface irradiating section, wherein
the light emitting modules are connected in parallel each other, the light emitting modules each having a plurality of light emitting devices connected in series each other and a single individual current control device, and
the individual current control device has a greater resistance value than a load impedance of the light emitting device at a predetermined temperature or less, and has a smaller resistance value than the load impedance of the light emitting device at a temperature exceeding the predetermined temperature.

3. The optical treatment apparatus according to claim 1 or 2, wherein the light emitting device is a near infrared light emitting diode.

4. The optical treatment apparatus according to claim 1 or 2, wherein the individual current control device is a thermistor having a characteristic that a resistance value of the thermistor is decreased with a rise in a temperature.

5. The optical treatment apparatus according to claim 4, wherein the thermistor is a CTR (Critical Temperature Coefficient) thermistor.

6. The optical treatment apparatus according to claim 1 or 2, wherein the individual current control device is disposed in the vicinity of irradiating target surface of the light emitting device which is connected in parallel with the individual current control device, and disposed in a position which is not directly irradiated with a light emitted from each of the light emitting device.

7. The optical treatment apparatus according to claim 6, wherein the individual current control device is disposed on an outer periphery of a light irradiating region of the light emitting device which is connected in parallel with the individual current control device.

8. The optical treatment apparatus according to claim 7, wherein the light emitting module has a heat insulator provided between the light emitting device and the individual current control device.

9. An optical treatment apparatus comprising: a surface irradiating section configured by connecting a plurality of light emitting module assemblies in parallel each other, the light emitting module assemblies each having a plurality of light emitting modules connected in series; and a current supplying section that supplies a current to the surface irradiating section, wherein
the light emitting module has a light emitting device and an individual current control device which are connected to each other in parallel, and
the individual current control device has a greater resistance value than a load impedance of the light emitting device at a predetermined temperature or less and having a smaller resistance value than the load impedance of the light emitting device at a temperature exceeding the predetermined temperature.

10. An optical treatment apparatus comprising: a surface irradiating section configured by connecting a plurality of light emitting module assemblies in parallel each other, the light emitting module assemblies each having a plurality of light emitting modules connected in series each other; and a current supplying section that supplies a current to the surface irradiating section, wherein
the light emitting module has a plurality of light emitting devices connected in series each other and a single individual current control device which is connected in parallel with the plurality of light emitting devices,
the individual current control device has a greater resistance value than a load impedance of the light emitting device at a predetermined temperature or less, and has a smaller resistance value than the load impedance of the light emitting device at a temperature exceeding the predetermined temperature.

11. The optical treatment apparatus according to claim 9 or 10, wherein the light emitting device is a near infrared light emitting diode.

12. The optical treatment apparatus according to claim 9 or 10, wherein the individual current control device is a thermistor having a characteristic that a resistance value of the thermistor is decreased with a rise in a temperature.

13. The optical treatment apparatus according to claim 12, wherein the thermistor is a CTR (Critical Temperature Coefficient) thermistor.

14. The optical treatment apparatus according claim 9 or 10, wherein the individual current control device is disposed in the vicinity of irradiating target surfaces of the light emitting device which is connected in parallel with the individual current control device, and disposed in a position which is not directly irradiated with a light emitted from the light emitting device.

15. The optical treatment apparatus according to claim 14, wherein the individual current control device is disposed on an outer periphery of a light irradiating region of the light emitting device which is connected in parallel with the individual current control device.

16. The optical treatment apparatus according to claim 15, wherein the light emitting module has a heat insulator provided between the light emitting device and the individual current control device.

17. The optical treatment apparatus according to claim 9 or 10, wherein the light emitting module assembly has a current limiting device connected to the light emitting module in series.
